(19) United States
(12) Patent Application Publication
(10) Pub. No.: US 2026/0284394 A1
(43) Pub. Date: Sep. 24, 2026

(54) SYSTEMS AND METHODS FOR SPINAL PATTERN STIMULATION

(71) Applicant: Boston Scientific Neuromodulation Corporation, Valencia, CA (US)

(72) Inventors: Rosana Esteller, Santa Clarita, CA (US); Tianhe Zhang, Studio City, CA (US); Satya Venkata Sandeep Avvaru, Houston, TX (US)

(73) Assignee: Boston Scientific Neuromodulation Corporation, Valencia, CA (US)

(21) Appl. No.: 19/575,292

(22) Filed: Mar. 23, 2026

Related U.S. Application Data
(60) Provisional application No. 63/776,642, filed on Mar. 24, 2025.

(51) Int. Cl. A61N 1/36

(52) U.S. Cl. CPC A61N 1/36062; A61N 1/36071; A61N 1/36171; A61N 1/36175

(57) ABSTRACT

Methods and systems for providing electrical stimulation to a patient’s spinal cord to treat at least inflammatory pain using an IPG system comprising one or more leads implanted in the patient’s spinal column, each lead comprising a plurality of electrodes, the method comprising delivering a first electrical stimulation signal at a first frequency between about 2 Hertz (Hz) to about 150 Hz to a patient's spinal cord; and delivering a second electrical stimulation signal at a second frequency between 200 Hz to about 1000 Hz to the patient's spinal cord concurrently with the first electrical stimulation signal.

### CROSS REFERENCE TO RELATED APPLICATIONS

This application claims the benefit of U.S. Provisional Patent Application Serial No. 63/776,642 filed on March 24, 2025, the disclosure of which is incorporated herein by reference.

### FIELD OF INVENTION

The present invention relates generally to medical device systems, and more particularly to neurostimulator systems operable to provide spinal pattern simulation.

### BACKGROUND

Spinal cord stimulation (SCS) therapy has emerged as an established treatment modality for managing chronic pain conditions through the delivery of electrical pulses to targeted areas of the spinal cord. The therapy operates by applying controlled electrical stimulation patterns through implanted electrodes positioned in the epidural space to modulate neural activity. Modern SCS systems incorporate advanced programming capabilities that enable the delivery of various stimulation frequencies and patterns, allowing for customized treatment approaches.

Chronic pain conditions can manifest through different mechanisms, including neuropathic, mechanical, and inflammatory pathways. These conditions may affect various parts of the body, from facial and head regions to peripheral locations such as wrists, knees, shoulders, and lower back. Typical implantable medical devices are indicated for use to treat neuropathic pain, not inflammatory pain. For instance, spinal cord stimulation (SCS) is typically employed to treat neuropathic pain (e.g., chronic neuropathic pain). Some traditional SCS approaches have primarily focused on treating neuropathic pain through single-frequency stimulation patterns. Some current implantable neurostimulation systems typically deliver continuous stimulation patterns that can lead to neural habituation, requiring increased stimulation intensities over time. New and alternative solutions directed to treatment of inflammatory pain are desired.

### OVERVIEW

Modulation for conventional SCS can be adjusted to map the paresthesia over the region of pain such as neurological pain. In other words, even under the optimum parameters the patient may feel the pain (e.g., neurological pain) in the affected area replaced by a tingling sensation and/or another sensation. Thus, it may be desirable to provide paresthesia-free modulation (e.g., sub-perception therapy) where a patient does not perceive the delivery of the modulation energy for sub-perception modulation.

The prevalence of inflammatory pain conditions and mixed pain conditions (e.g., inflammatory pain and neuropathic pain) is significant, with conditions such as rheumatoid arthritis affecting 0.5 to 1% of the population, multiple sclerosis affecting approximately 1 million individuals in the US, various types of cancer, and fibromyalgia impacting about 2% of the US population. Yet despite the significant prevalence of inflammatory pain conditions and mixed pain conditions, these types of conditions remain difficult to address. For instance, some approaches may utilize multiple distance medical devices (e.g., one type of implantable medical device such as an SCS system configured to treat neuropathic pain and another type of implantable medical device such as a vagus nerve stimulator configured to treat inflammatory pain). Such approaches may be ineffective (e.g., at least in terms of efficacy of treatment of inflammatory pain), complex, costly, and/or otherwise undesirable (e.g., may numerous implant/explant procedures over an operational lifetime of the implantable medical devices).

Additionally, approximately 56 percent (%) of patients with traditional SCS eventually undergo explantation, with about 23% due to loss of therapeutic performance. The challenge of maintaining long-term efficacy while preventing neural habituation has been a significant limitation of existing SCS approaches. Some examples may be configured to prevent habituation.

Hence, the present disclosure provides a new approach to treating inflammatory pain which was previously not treatable via SCS. Moreover, the present disclosure provides a system including a single medical device (e.g., a single implantable IPG) that is configured to treat mixed chronic, thereby reducing the cost, difficulties, and/or complexities typically associated with previous approaches to treat mixed chronic pain. That is, the systems and methods herein utilize pattern stimulation (e.g., spinal pattern stimulation) delivered concurrently at sub-perception thresholds. As used herein the term concurrently refers to the delivery of electrical stimulation by two distinct stimulation programs such that their respective stimulation events occur within overlapping time periods. This concurrently stimulation delivery includes scenarios where one program begins before or after the other while still overlapping in time, as well as cases where both programs deliver stimulation at the exact same moment (simultaneously).

In some examples, the system delivers a combination waveform comprised of a low-frequency stimulation (e.g., 40-60 Hertz (Hz)) combined with a high-frequency stimulation (e.g., 400-1000 Hz) through a spinal cord stimulator using specific electrode configurations. The stimulation is delivered at amplitudes between 20-80% of the paresthesia threshold (e.g., is delivered as paresthesia-free stimulation), with specific pulse widths for each frequency component (80-200 microseconds for low frequency; 20-80 microseconds for high frequency). Yet, the combination waveform can have a low duty cycle (e.g., a duty cycle of less than about 70 percent, less than about 30 percent, etc.) that can be reconfigured (e.g., can be reduced based on patient feedback, etc.) over time to address patient habituation or plasticity. For instance, the system can be configured with controlled ON/OFF cycling patterns, such as 2 hours ON followed by 3-6 hours OFF, which can help prevent or account from neural habituation while also maintaining therapeutic efficacy. The system can incorporate adaptive features based on patient activity, time of day, and sleep patterns, while maintaining energy efficiency through optimized cycling patterns. Clinical observations have demonstrated sustained pain relief in patients with inflammatory conditions, particularly those with rheumatoid arthritis, using this spinal pattern stimulation approach. As such, the systems and methods herein pertain to paresthesia-free stimulation using a combination waveform to address new indications (e.g., inflammatory pain) previously unresolvable by SCS with low duty cycle that is reconfigurable over time to address patient habituation or plasticity.

An illustrative and non-limiting example takes the form of a method for providing electrical stimulation to a patient’s spinal cord to treat at least inflammatory pain using an IPG system comprising one or more leads implanted in the patient’s spinal column, each lead comprising a plurality of electrodes, the method comprising: delivering a first electrical stimulation signal at a first frequency between about 2 Hertz (Hz) to about 150 Hz to a patient's spinal cord; and delivering a second electrical stimulation signal at a second frequency between 200 Hz to about 1000 Hz to the patient's spinal cord concurrently with the first electrical stimulation signal.

Alternatively or in addition to any of the examples herein, wherein both the first and second electrical stimulation signals are delivered at amplitudes that are less than about 80 percent of a paresthesia threshold.

Alternatively or in addition to any of the examples herein, wherein both the first and second electrical stimulation signals are delivered at amplitudes between about 20 to about 80 percent of a paresthesia threshold.

Alternatively or in addition to any of the examples herein, wherein both the first and second electrical stimulation signals are delivered at amplitudes between about 20 to about 40 percent of a paresthesia threshold.

Alternatively or in addition to any of the examples herein, wherein a ratio of the first frequency to the second frequency is in a range from about 1:10 to about 1:50.

Alternatively or in addition to any of the examples herein, wherein the first frequency is in a range from about 20 Hz to about 90 Hz.

Alternatively or in addition to any of the examples herein, wherein the first frequency is in a range from about 40 Hz to about 60 Hz.

Alternatively or in addition to any of the examples herein, wherein the second frequency is in a range from about 400 Hz to about 600 Hz.

Alternatively or in addition to any of the examples herein, further comprising cycling the concurrent delivery of the first and second electrical stimulation signals at a duty cycle that is less than about 0.8.

Alternatively or in addition to any of the examples herein, further comprising cycling the concurrent delivery of the first and second electrical stimulation signals at a duty cycle in the range from about 0.3 to about 0.7.

Alternatively or in addition to any of the examples herein, further comprising cycling the concurrent delivery of the first and second electrical stimulation signals at a duty cycle in the range from about 0.3.

Alternatively or in addition to any of the examples herein, wherein a pulse width of the first electrical stimulation signal is larger than a pulse width of the second electrical stimulation signal.

Alternatively or in addition to any of the examples herein, the first electrical stimulation signal has a pulse width between about 80 to about 200 microseconds; and the second electrical stimulation signal has a pulse width between about 20 to about 80 microseconds.

Alternatively or in addition to any of the examples herein, wherein an area in the spinal cord stimulated by the first and second signals overlaps a dermatome associated with the inflammatory pain.

Alternatively or in addition to any of the examples herein, further comprising initiating delivery of the first electrical stimulation signal and the second electrical stimulation signal, ceasing delivery of the first electrical stimulation signal and the second electrical stimulation signal, altering a parameter of the first electrical stimulation signal and the second electrical stimulation signal, or any combination thereof, based on: accelerometer data; a time of day; a patient or clinician input; or any combination thereof.

Alternatively or in addition to any of the examples herein, further comprising delivering the first electrical stimulation signal and the second electrical stimulation signals as active recharge electrical stimulation signals, as square waves, or both.

Another illustrative and non-limiting example takes the form of a spinal cord stimulation system comprising: a lead including a plurality of electrodes; and an implantable pulse generator configured to: generate a first electrical stimulation signal at a first frequency between about 2 to about 150 Hz and a pulse width between about 80 to about 200 microseconds; generate a second electrical stimulation signal at a second frequency between 400-1000 Hz and a pulse width between about 20 to about 80 microseconds; deliver, via the first electrical stimulation signal and the second electrical stimulation signal concurrently to a patient's spinal cord at amplitudes between about 20 to about 80 % of a paresthesia threshold of the patient to treat at least inflammatory pain of the patient; and cycle the concurrent delivery according to a duty cycle in a range from about 0.2 to about 0.8.

Alternatively or in addition to any of the examples herein, wherein at least two of the plurality of electrodes are configured as cathodic electrodes and at least two of the electrodes are configured as anodic electrodes.

Another illustrative and non-limiting example takes the form of A non-transitory computer-readable medium having instructions stored thereon to cause circuitry in a computing device to: generate a first electrical stimulation signal at a first frequency between about 2 to about 150 Hz and a pulse width between about 80 to about 200 microseconds; generate a second electrical stimulation signal at a second frequency between 400-600 Hz and a pulse width between about 20 to about 80 microseconds; deliver, via the plurality of electrodes, the first electrical stimulation signal and the second electrical stimulation signal concurrently to a patient's spinal cord at amplitudes between about 20 to about 40 % of a paresthesia threshold of the patient to treat mixed pain of the patient, wherein a delivery location of the first electrical signal and a delivery location of the second electrical signal overlap and are configured to target the same dermatome associated with the mixed pain; and cycle the concurrent delivery according to a duty cycle in a range from about 0.2 to about 0.8.

Alternatively or in addition to any of the examples herein, wherein the first electrical stimulation signal and the second electrical stimulation signal are configured as a series of square wave pulses.

Alternatively or in addition to any of the examples herein, wherein the first electrical stimulation signal and the second electrical stimulation signal are delivered for two hours or less per day.

Alternatively or in addition to any of the examples herein, wherein the IPG is configured to deliver, via at least four of the plurality of electrodes, the first electrical stimulation signal and the second electrical stimulation signal concurrently.

Alternatively or in addition to any of the examples herein, wherein at least four electrodes include at least two cathodic electrodes and at least two anodic electrodes; and the at least two cathodic electrodes are spaced at least one electrode away from the at least two anodic electrodes.

This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document. Such embodiments are demonstrative and not intended to be exhaustive or exclusive embodiments of the present subject matter.

FIGS. 1A and 1B respectively show an Implantable Pulse Generator (IPG) in plan and cross-sectional views.

FIG. 2 shows a hand-held external controller for communicating with an IPG.

FIG. 3 shows a clinician programming system for communicating with an IPG or an External Trial Stimulator (ETS).

FIGS. 4A and 4B illustrate a spinal cord and related neural anatomy.

FIGS. 5A-5E illustrate leads configurable for a spinal pattern stimulation waveform.

FIG. 5F illustrates a spinal pattern stimulation waveform.

FIG. 6 illustrates a method of spinal pattern stimulation for treatment of at least inflammatory pain.

FIG. 7 illustrates a block diagram illustrating a machine in the example form of a computer system, within which a set or sequence of instructions may be executed to cause the machine to perform any one of the methodologies discussed herein, according to an example embodiment.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter.

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and Deep Brain Stimulators (DBS) to treat motor and psychological disorders, sacral nerve stimulators to treat overactive bladder or bladder emptying problems, vagus nerve stimulators to treat epilepsy, trigeminal nerve stimulators to treat migraine and epilepsy, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any Implantable Medical Device (IPG) or in any IPG system.

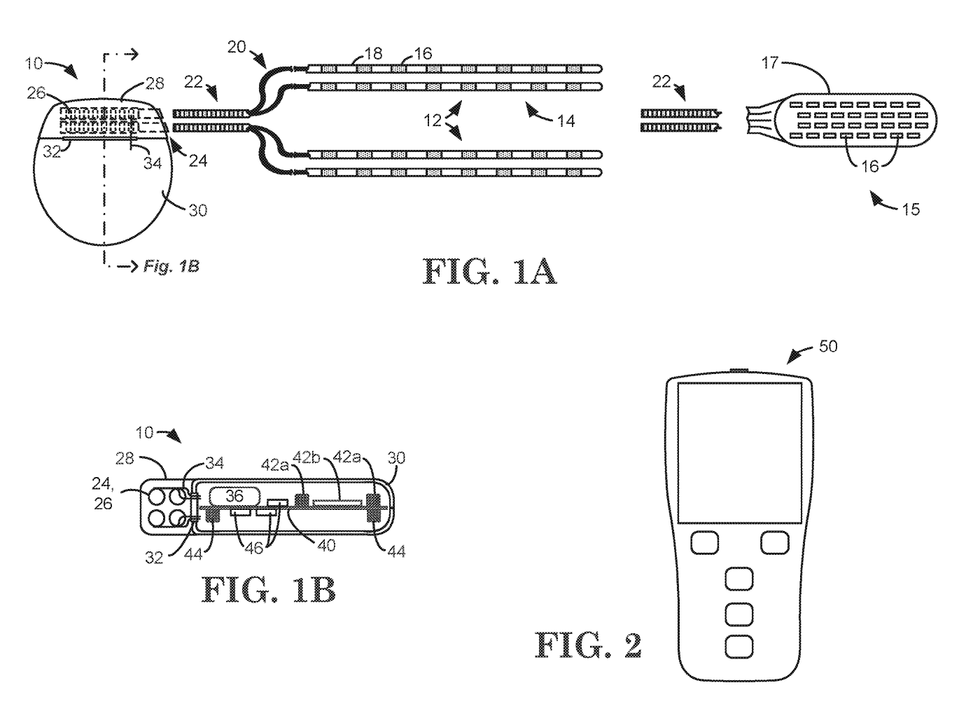

An SCS system typically includes an Implantable Pulse Generator (IPG) 10 shown in plan and cross-sectional views in FIGS. 1A and 1B. The IPG 10 includes a biocompatible device case 30 is configured for implantation in a patient's tissue that holds the circuitry and battery 36 (FIG. 1B) necessary for the IPG to function. The IPG 10 is coupled to electrodes 16 via one or more electrode leads 14 that form an electrode array 12. The electrodes 16 are configured to contact a patient's tissue and are carried on a flexible body 18, which also houses the individual lead wires 20 coupled to each electrode 16. The lead wires 20 are also coupled to proximal contacts 22, which can be inserted into lead connectors 24 fixed in a header 28 on the IPG 10, which header can comprise an epoxy for example. Once inserted, the proximal contacts 22 connect to header contacts 26 in the lead connectors 24, which are in turn coupled by electrode feedthrough pins 34 through an electrode feedthrough 32 to circuitry within the case 30 (connection not shown).

In the illustrated IPG 10, there are thirty-two lead electrodes (E1-E32) split between four leads 14 (referred to as percutaneous leads), with the header 28 containing a 2×2 array of lead connectors 24 to receive the leads' proximal ends. However, the number of leads and electrodes in an IPG is application specific and therefore can vary. In a SCS application, the electrode leads 14 are typically implanted proximate to the dura in a patient's spinal cord, and when a four-lead IPG 10 is used, these leads can be split with two on each of the right and left sides. The proximal contacts 22 are tunneled through the patient's tissue to a distant location such as the buttocks where the IPG case 30 is implanted, at which point they are coupled to the lead connectors 24. As also shown in FIG. 1A, one or more flat paddle leads 15 can also be used with IPG 10, and in the example shown thirty-two electrodes 16 are positioned on one of the generally flat surfaces of the head 17 of the paddle lead, which surface would face the dura when implanted. In other IPG examples designed for implantation directly at a site requiring stimulation, the IPG can be lead-less, having electrodes 16 instead carried by the case of the IPG for contacting the patient's tissue.

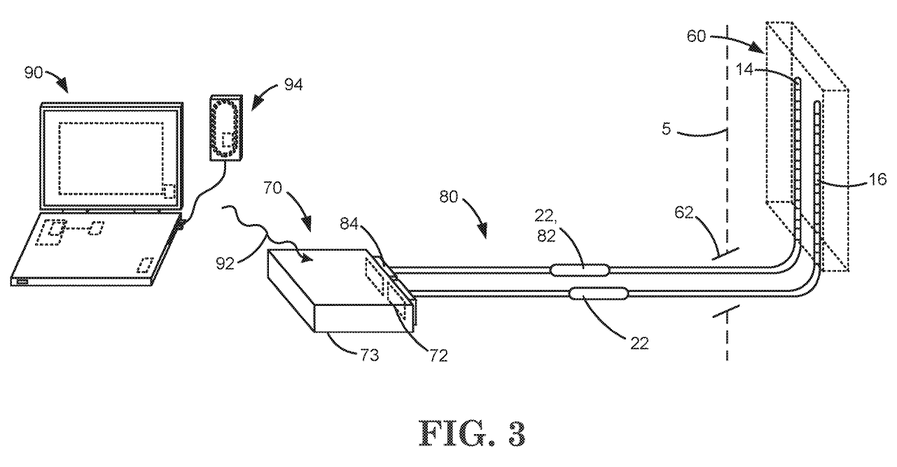

As shown in the cross section of FIG. 1B, the IPG 10 includes a printed circuit board (PCB) 40. Electrically coupled to the PCB 40 are the battery 36, which in this example is rechargeable; other circuitry 46 coupled to top and/or bottom surfaces of the PCB 40, including a microcontroller and associated memory storing controller-readable instructions for operation of the IPG 10 (execution of which may result in performance of the illustrative methods described herein) and/or other control circuitry necessary for IPG operation; a telemetry antenna-42 a and/or 42b—for wirelessly communicating data with an external controller 50 (FIG. 2); a charging coil 44 for wirelessly receiving a magnetic charging field from an external charger (not shown) for recharging the battery 36; and the electrode feedthrough pins 34 (connection to circuitry not shown). If battery 36 is permanent and not rechargeable, charging coil 44 would be unnecessary.

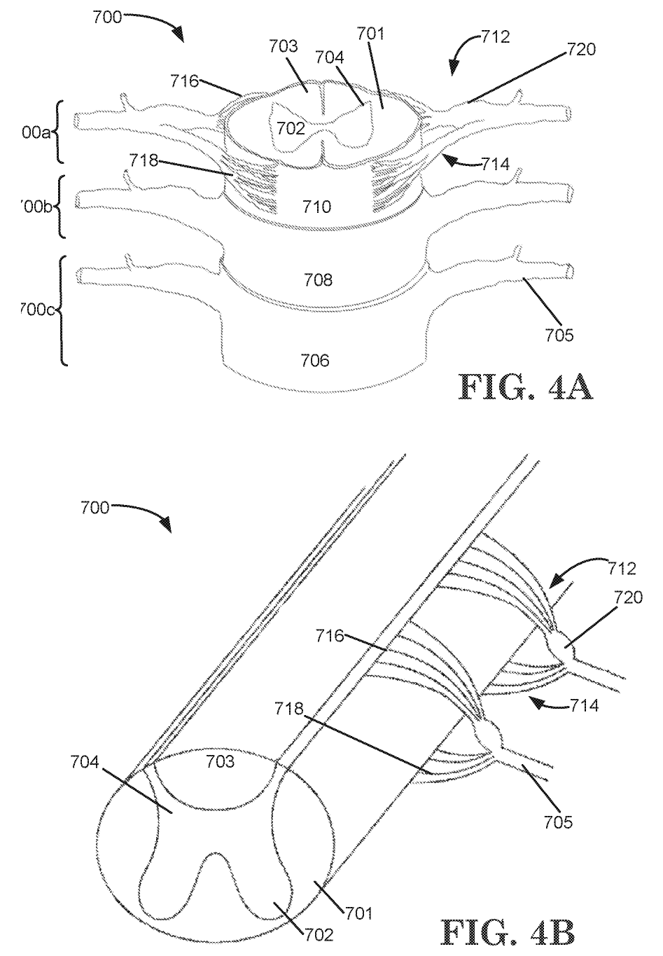

The IPG 10 also includes one or more antennas 42 a and 42 b for transcutaneously communicating with external programming devices, such as a patient external controller 50 (FIG. 2), or a clinician programmer 90 (FIG. 3). Antennas 42 a and 42 b are different in shape and in the electromagnetic fields they employ. Telemetry antenna 42 a comprises a coil, which can bi-directionally communicate with an external device via a magnetic induction communication link. Telemetry antenna 42 b comprises a short-range Radio-Frequency (RF) antenna that operates in accordance with a short-range RF communication standard, such as Bluetooth, BLE, NFC, Zigbee, WiFi (802.11x), and the Medradio and/or or the Medical Device Radiocommunications Service (MDRS).

The IPG 10 may include separate circuits 46, sometimes referred to as operational circuitry, including a microcontroller (which may also be implemented as part of a microprocessor if desired), configured to control operations of the IPG 10 at a high level. The microcontroller may include memory for storing operational instructions in a non-transitory media, such as a Flash memory, RAM, ROM, etc. The IPG 10 also includes stimulation circuitry. At a high level, the stimulation circuitry may include a plurality of current sources and current sinks (for a current-controlled system; a plurality of voltage sources may be used in voltage-controlled systems instead), and control circuitry including for example one or more analog ASICs, as well as switch arrays that implement steering instructions and/or electrode selections. US Patent 10,716,932 provides illustrative examples and details for both current and planned future implementations of the stimulation circuitry and is incorporated herein by reference. As noted, other designs for stimulation circuitry may be used. Multiple sources (current or voltage) allow control over individual electrodes on the lead (FIG. 1A) to create shaped electrical fields adapted to selectively modulate or otherwise affect specific neural tissue.

Implantation of IPG 10 in a patient is normally a multi-step process, as explained with reference to FIG. 3. A first step involves implantation of the distal ends of the lead(s) 14 or 15 with the electrodes 16 into the spinal column 60 of the patient through a temporary incision 62 in the patient's tissue 5 (Only two leads 14 with sixteen total electrodes 16 are shown in FIG. 3 for simplicity). The proximal ends of the leads 14 or 15 including the proximal contacts 22 extend externally from the incision 62 (i.e., outside the patient) and are ultimately connected to an External Trial Stimulator (ETS) 70. The ETS 70 is used during a trial stimulation phase to provide stimulation to the patient, which may last for two or so weeks for example. To facilitate the connection between the leads 14 or 15 and the ETS 70, ETS extender cables 80 may be used that include receptacles 82 (similar to the lead connectors 24 in the IPG 10) for receiving the proximal contacts 22 of leads 14 or 15, and connectors 84 for meeting with ports 72 on the ETS 70, thus allowing the ETS 70 to communicate with each electrode 16 individually. Once connected to the leads 14 or 15, the ETS 70 can then be affixed to the patient in a convenient fashion for the duration of the trial stimulation phase, such as by placing the ETS 70 into a belt worn by the patient (not shown). ETS 70 includes a housing 73 for its control circuitry, antenna, etc., which housing 73 is not configured for implantation in a patient's tissue.

The ETS 70 essentially mimics operation of the IPG 10 to provide stimulation to the implanted electrodes 16 and thus contains a battery within its housing along with stimulation and communication circuitry similar to that provided in the IPG 10. Thus, the ETS 70 allows the effectiveness of stimulation therapy to be verified for the patient, such as whether therapy has alleviated the patient's symptoms (e.g., pain). Trial stimulation using the ETS 70 further allows for the determination of particular stimulation program(s) that seems promising for the patient to use once the IPG 10 is later implanted into the patient. A stimulation program may include stimulation parameters that specify for example: which of the electrodes 16 are to be active and used to issue stimulation pulses; the polarity of those active electrodes (whether they are to act as anodes or cathodes); the current or voltage amplitude (A) of the stimulation pulses; the pulse width (PW) of the stimulation pulses; the frequency (f) of the stimulation pulses; the duty cycle (DC) of the stimulation pulses (i.e., the percentage of time that the pulses are asserted relative to the period of the pulses) the shape of the stimulation waveform (e.g., one or more square pulses, one or more ramped pulses, one or more sinusoidal pulses, or even non-pulse-based waveforms, etc.); and other parameters related to issuing a burst of pulses, such as the number of pulses; etc.

The stimulation program executed by the ETS 70 can be provided or adjusted via a wired or wireless link 92 (wireless shown) from a clinician programmer 90. As shown, the clinician programmer 90 comprises a computer-type device and may communicate wirelessly with the ETS 70 via link 92, which link may comprise magnetic inductive or short-range RF telemetry schemes as already described. Should the clinician programmer 90 lack a communication antenna, a communication head or wand 94 may be wired to the computer which has a communication antenna. Thus, the ETS 70 and the clinician's programmer 90 and/or its communication head 94 may include antennas compliant with the telemetry scheme chosen. Clinician programmer 90 may be as described in U.S. Patent Application Publication 2015/0360038. External controller 50 (FIG. 2) may also communicate with the ETS 70 to allow the patient means for providing or adjusting the ETS 70's stimulation program.

At the end of the trial stimulation phase, a decision is made whether to abandon stimulation therapy, or whether to provide the patient with a permanent IPG 10 such as that shown in FIGS. 1A and 1B. Should it be determined that stimulation therapy is not working for the patient, the leads 14 or 15 can be explanted from the patient's spinal column 60 and incision 62 closed in a further surgical procedure. By contrast, if stimulation therapy is effective, IPG 10 can be permanently implanted in the patient as discussed above. (“Permanent” in this context generally refers to the useful life of the IPG 10, which may be from a few years to a few decades, at which time the IPG 10 would need to be explanted and a new IPG 10 implanted). Thus, the IPG 10 would be implanted in the correct location (e.g., the buttocks) and connected to the leads 14 or 15, and then temporary incision 62 can be closed and the ETS 70 dispensed with. The result is fully implanted stimulation therapy solution. If a particular stimulation program(s) had been determined during the trial stimulation phase, it/they can then be programmed into the IPG 10, and thereafter modified wirelessly, using either the external programmer 50 or the clinician programmer 90.

This document discusses various techniques that can generate, determine, or monitor programming values or treatment effects of an implantable electrical neurostimulation device, in connection with the treatment of pain or related physiological conditions in a human subject (e.g., a patient). For instance, various embodiments described herein involve neural modulation. Examples of neural modulation include spinal cord stimulation (SCS), deep brain stimulation (DBS), peripheral nerve stimulation (PNS), vagus nerve stimulation (VNS), and sacral nerve stimulation for overactive bladder (OAB).

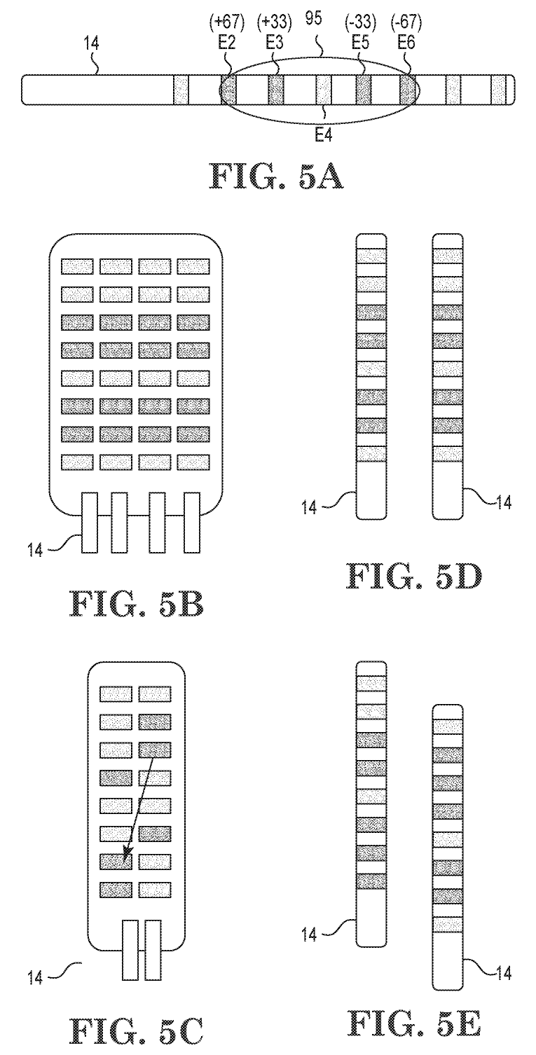

A brief description of the physiology of the spinal cord is provided herein to assist the reader with regard to embodiments involving SCS. FIGS. 4A and 4B illustrate, by way of example, a portion of a spinal cord 700 including white matter 701 and gray matter 702 of the spinal cord. The gray matter 702 includes cell bodies, synapse, dendrites, and axon terminals. Synapses are located in the gray matter. White matter 701 includes myelinated axons that connect gray matter areas. A typical transverse section of the spinal cord includes a central “butterfly” shaped central area of gray matter 702 substantially surrounded by an ellipse-shaped outer area of white matter 701. The white matter of the dorsal column (DC) 703 includes mostly large, myelinated axons that form afferent fibers that run in an axial direction. The dorsal portions of the “butterfly” shaped central area of gray matter are referred to as dorsal horns (DH) 704. In contrast to the DC fibers that run in an axial direction, DH fibers can be oriented in many directions, including laterally with respect to the longitudinal axis of the spinal cord.

Referring to FIG. 4A, the spinal cord is enclosed within three layers of tissue, collectively called the meninges. The outer layer of the meninges, called the dura mater 706, is shown in spinal cord segment 700c. The dura mater has been removed in spinal cord segment 700b to reveal the middle meninges, called the arachnoid 708. The innermost meninges, the pia mater 710, is shown in spinal cord segment 700a.

Examples of spinal nerves 705 are also illustrated. Upon removal of the meningeal layers, it is seen that each spinal nerve 705 splits into a dorsal root (DR) 712 and a ventral root 714, each of which comprise subdivisions referred to as rootlets. In FIG. 4A, the dorsal rootlets are labeled 716 and the ventral rootlets are labeled 718. The dorsal root also includes a structure called the dorsal root ganglion (DRG) 720, which comprises cell bodies of the afferent neurons. The dorsal root 712 contains afferent neurons, meaning that they carry sensory signals into the spinal cord, and the ventral root 714 functions as an efferent motor root. The dorsal and ventral roots join to form mixed spinal nerves 705.

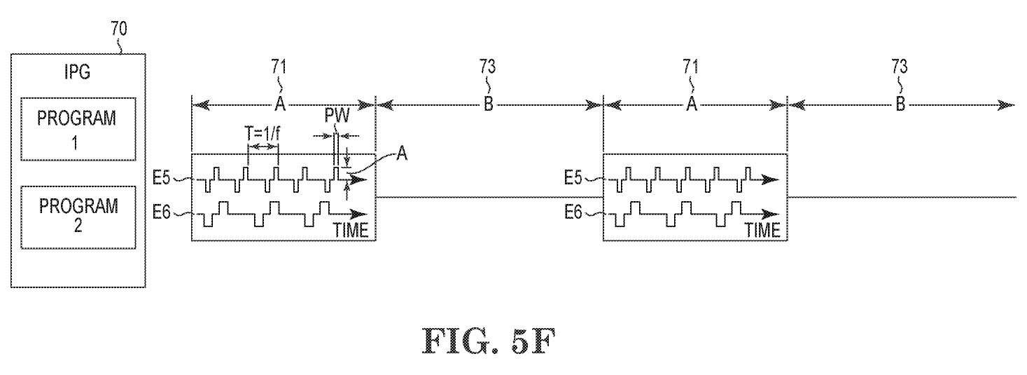

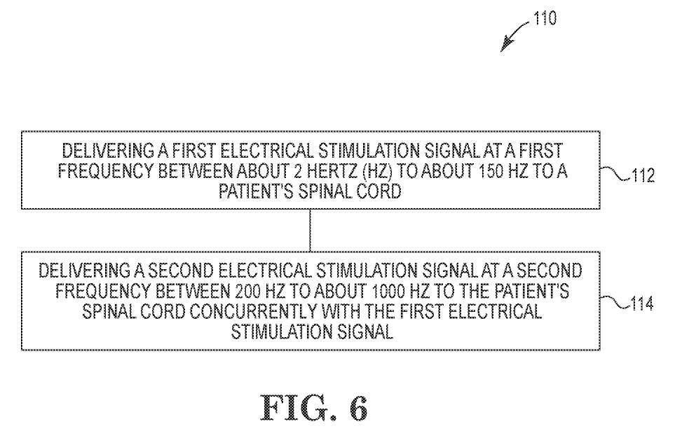

An example of stimulation pulses as prescribed by an example spinal pattern stimulation program and as executable by the IPG 10 or ETS 70. While FIG. 5A illustrates a particular arrangement and quantity of electrodes in the form of a 1 x 8 percutaneous lead 14 including a plurality of electrodes 16, any existing electrode design and/or combination of electrodes in any quantity of rows and columns of electrodes including, but not limited to, 1 x 8, 1 x 16, 4 x 4, or 4 x8 configurations of paddle, angled, and/or percutaneous leads may be employed. For instance, the systems and methods herein may use an individual 4 x 8 paddle lead 14, an individual angled 2 x 8 lead 14, two 1 x 8 percutaneous leads 14, or two staggered (e.g., uneven) 1 x 8 percutaneous leads 14, as illustrated in FIGS. 5B-5E, respectively.

Returning to FIG. 5A, the plurality of electrodes 16 can include electrodes E2, E3 that are selected or configured as anodes and electrodes E5, E6 that are selected or configured as cathodes on the example 1 x 8 percutaneous lead 14. The electromagnetic fields can be fractionalized in any manner. For instance, as the anodic electrodes can be fractionalized (e.g., monotonic fractionalization) with the same or different values than the corresponding values in the cathodic electrodes. For instance, E2, E3 can be fractionalized as 67/33 and E5, E6 can be fractionalized as 33/67, as illustrated in FIG. 5A. This is merely an example and the use of alternate values or configurations is possible. For instance, while the weights or fractionalization of the electrodes in FIG. 5A is equal (e.g., is 33/67 in both the anodic and cathodic electrodes), in some embodiments different or unequal fractionalizations may be employed. In the example stimulation program shown and considering only the first phase of the biphasic pulses, electrodes E5 and E6 are selected to operate as a cathode (−), and electrodes E2 and E3 are selected to operate as anodes (+). Such stimulation is usually referred to as quadpolar stimulation. Quadpolar and bipolar stimulation are some of the preferred modes of providing stimulation, particularly in an SCS application, because neural fibers in the dorsal column are activated proximate to the cathode while using the anodes as flanking regions. Anodic activation exhibits a higher threshold compared to cathodal stimulation. An embodiment to use anodic activation can be used by placing the cathode electrode in the device can.

In the example shown, the pulses are defined with respect to a total anodic and cathodic current (collectively, Itot) that the electrodes will provide at any given time. This is desirable so that the patient's tissue will not receive a net amount of charge. The sole cathode electrode E4 provides all the total cathodic current (−Itot), and so provides 100*−Itot, or −A. The two anode electrodes E2 and E3 together issue the total anodic current (+Itot). The anode electrodes can issue any anodic currents that together will equal the total cathodic current. It is assumed that these stimulation programs, namely a combination thereof in the form of spinal pattern stimulation programs, have been chosen as one that generally provides good therapeutic results for a particular patient.

That is, the cathodic and anodic electrodes may be configured to deliver the pattern stimulation, as described herein. For instance, one or more of the cathodic electrodes and one or more of the anodic electrodes may be configured to deliver a first electrical stimulation signal, while one or more of the cathodic electrodes and one or more of the anodic electrodes may be configured to deliver a second electrical stimulation signal. That is, the electrodes 16 can include at least two electrodes configured as cathodic electrodes and at least two different electrodes configured as anodic electrodes to permit the concurrent delivery of the first electrical stimulation signal and the second electrical stimulation signal.

As mentioned, electrodes E2, E3, E5, and E6 on lead 14 are used as two sets of electrodes that together produce pulses in a quadpolar stimulation program, with E2 and E3 comprising anodes (+; or source of current) and E5 and E6 comprising cathodes (−; or sink of current). Such stimulation produces an electromagnetic (EM) field in a volume 95 of the patient's tissue around the selected electrodes. Some of the neural elements within the EM field volume 95 will be recruited and fire, particularly those proximate to the cathodic electrodes E5 and E6. Thus, the sum of the neural elements firing within volume 95 will mask signals indicative of pain in the form of inflammatory pain or mixed pain in an SCS application, thus providing the desired therapy.

In some embodiments, at least one electrode (e.g., E4) positioned between the cathodic electrodes and the anodic electrodes can be configured as a reference electrode or another type of electrode other than a cathodic or anodic electrode. For instance, in some embodiments, the IPG 10 can be configured to deliver, via at least four of the plurality of electrodes, the first electrical stimulation signal and the second electrical stimulation signal concurrently. In such embodiments, at least four electrodes can include at least two cathodic electrodes and at least two anodic electrodes and the at least two cathodic electrodes can space (e.g., longitudinally along a length of the lead) at least one electrode away from the at least two anodic electrodes. Having at least one electrode (e.g., a reference or spacer electrode) positioned between the cathodic and anodic electrodes, unlike some other approaches where the cathodic and anodic electrodes are directly adjacent, can promote aspects herein such as promoting delivery of the first electrical stimulation signal and the second electrical stimulation signal to a larger area (i.e., maximizing a size of the electromagnetic field). This relatively large electromagnetic field can promote delivery of the stimulation to an area in the spinal cord that is sufficiently large to treat at least inflammatory pain and/or treat mixed pain. For instance, both the first electrical stimulation signal and the second electrical stimulation signal may overlap a dermatome associated with mixed pain (e.g., inflammatory pain and another type of pain such as neuropathic pain).

As mentioned, the first electrical stimulation signal (e.g., program 1) and the second electrical stimulation signal (e.g., program 2) can be delivered by the IPG 10 or ETS 70, described herein, to one or more leads concurrently (over the same time period). For instance, FIG. 5F illustrates the waveforms of the stimulation pulses delivered concurrently by the IPG 10 or ETS 70 (e.g., delivered to electrodes E5 and E6), which correspond to the second electrical stimulation signal and the first electrical stimulation signal, respectively. While references are made to first and second programs and/or waveforms herein, such references are made for illustrative purposes and do not necessarily limit the aspects described herein to a particular arrangement or order. The stimulation program is defined by various stimulation parameters to form stimulation pulses, such as which electrodes are active for stimulation, the polarity of those electrodes, the amplitude at selected electrodes, pulse width at each pulse phase, pulse frequency, cycling, and stimulation waveform shape (square pulses in the example shown), although these parameters are not all labeled in FIG. 5F.

Concurrent delivery of the first electrical stimulation signal and the second electrical stimulation signal as sub-perception stimulation levels, as detailed herein, can yield synergistic benefits (e.g., permits that treatment of inflammatory pain or mixed pain) as compared to using either stimulation signal (e.g., either frequency) alone e.g., as is typically employed. Some SCS embodiments deliver sub-perception therapy that is therapeutically effective to treat pain, for example, but wherein the patient does not sense the delivery of the modulation field (e.g. paresthesia). Sub-perception therapy can be delivered at any frequency below 1 Hz or above 1 Hz and may include higher frequency modulation (e.g. about 1000 Hz or above) of the spinal mechanisms producing pain relief, likely by interfering with the transmission of pain signals. Some embodiments herein selectively modulate DH neural targets or DR neural targets over DC targets to provide sub-perception therapy. Embodiments of sub-perception therapy are described in U.S. Patent Application Publication Nos. 2016/0082262, 2016/0082251, and 2016/0082268, the contents of which are incorporated herein by reference.

In some embodiments, first electrical stimulation signal and the second electrical stimulation signal can be monophasic or biphasic. The pulse width (PW) could comprise the duration of either of the pulse phases individually as shown. The frequency (f) and amplitude (A) of the pulses is also shown. Beginning at 0 ms, electrode E5 is programmed to produce a first phase current (cathode) at the same time that electrode E6 is programmed to produce first phase current (cathode). However, the first electrical stimulation signal and the second electrical stimulation signal have different parameters (e.g., different frequencies), as detailed herein.

In some embodiments, the first electrical stimulation signal and the second electrical stimulation signal can be configured with a corresponding passive recharge or a corresponding active recharge phase. In a preferred embodiment, the first electrical stimulation signal and the second electrical stimulation signal can be configured with a corresponding active recharge phase, for instance, where the recharge amplitude shall be programmed to the opposite polarity and amplitude as the first phase. Using active recharge in this manner allows faster recharge while avoiding the charge imbalance that could otherwise occur, among other benefits.

The first electrical stimulation signal and the second electrical stimulation signal can be delivered concurrently for a first time period (e.g., an ON period), represented as element 71 in FIG. 5F. Immediately following the first time period 71, the first electrical stimulation signal and the second electrical stimulation can cease to be delivered for a second time period (e.g., an OFF period), represented as element 73. Immediately following the second time period 73, the first electrical stimulation signal and the second electrical stimulation can be delivered at the same parameters as previously delivered during the initial ON period. That is, the first electrical stimulation signal and the second electrical stimulation signal can be periodically delivered at a given duty cycle (e.g., a ratio of ON time to OFF time).

The duty cycle can be less than about 0.8. The use of the relatively low duty cycles herein can desirably reduce an amount of power consumption (e.g., extend an operational lifetime of a battery in an IPG) and/or can mitigate patient habitation to stimulation. For instance, the duty cycle can be less than about 0.8, less than about 0.7, less than about 0.6, less than about 0.5, less than about 0.4, or less than about 0.3. In some embodiments, the duty cycle is in the range from about 0.3 to about 0.8 or is in a range from about 0.3 to about 0.7. In some embodiments, the duty cycle can be about 0.8, about 0.7, about 0.6, about 0.5, about 0.4, or about 0.3. In some instances, the ON time period 71 can be about 1 hour, about 2 hours, or about three hours. In some instances, the OFF time period can be about 6 hours, about 8 hours, about 12 hours, about 18 hours, about 24 hours, about 36 hours, or about 48 hours. For instance, in some embodiments the ON time period 71 is about 2 hours and the OFF time period is about 3 hours or about 6 hours, among other possibilities. In some embodiments, the duty cycle can be adjusted (e.g., with a patient or physical programmer) after a period of time (e.g., after an initial wash-in or acclimation period).

A pulse width of the first electrical stimulation signal can be different than a pulse width of the second electrical stimulation signal. For instance, a pulse width of the first electrical stimulation signal can be larger than a pulse width of the second electrical stimulation signal. In some embodiments, the pulse width of the first electrical stimulation signal can be at least 1.5, at least 2.0, or at least 2.5 times larger than a pulse width of the second electrical stimulation signal. In some embodiments, the first electrical stimulation signal has a pulse width between about 80 to about 200 microseconds and the second electrical stimulation signal has a pulse width between about 20 to about 80 microseconds. In some embodiments, the pulse width of the first electrical stimulation signal can be about 150 microseconds, and the pulse width of the second electrical stimulation signal can be about 50 microseconds, among other possible values.

The first and second electrical stimulation signals can be delivered at amplitudes that are less than about 80 percent of a paresthesia threshold (e.g., a patient-specific paresthesia threshold identified during initial testing or otherwise identified). Stated differently, each of the first and second electrical stimulation signals can be delivered at amplitudes that are less than about 80 percent of a paresthesia threshold. For instance, both the first and second electrical stimulation signals can be delivered at amplitudes between about 20 to about 80 percent of a paresthesia threshold. In some embodiments, both the first and second electrical stimulation signals can be delivered at amplitudes between about 20 to about 40 percent of a paresthesia threshold, and yet the systems and methods herein can effectively treat at least inflammatory pain (e.g., treat inflammatory pain alone or mixed pain in the form of inflammatory pain along with another type of pain such as neuropathic pain).

The first frequency can be in a range between about 2 Hertz (Hz) to about 150 Hz. For instance, the first frequency can be about 2, about 4, about 6, about 8, about 10, 20, about 30 about 40, about 50, about 60, about 70, about 80, or about 90 Hz. In some embodiments, the first frequency can be in a range from about 20 to about 90 Hz or from about 40 Hz to about 60 Hz.

The second frequency can be in a range from about 200 Hz to about 1000 Hz. For example, the second frequency can be about 200, about 300 about 400, about 500, about 600, about 700, about 800, about 900 Hz, or about 1000 Hz. In some embodiments, the second frequency is in a range from about 400 Hz to about 600 Hz, about 300 Hz to about 600 Hz, or about 300 Hz to about 500 Hz.

In some embodiments, the first frequency is different (e.g., is lower than) the second frequency. For instance, a ratio of the first frequency of the first electrical stimulation signal to the second frequency of the second electrical stimulation signal can be in a range from about 1:500 to about 1:100, about 1:10 to about 1:50, or about 1:10 to about 3:20. For instance, the ratio of the first frequency to the second frequency can be about 1:500 (e.g., about 2 Hz to about 1000 Hz), about 1:100 (e.g., about 2 Hz to about 200 Hz), about 1:10 (e.g., about 40 Hz to about 400 Hz), about 3:20 (about 60 Hz to about 400 Hz), about 1:15 (about 40 Hz to about 600 Hz), or about 1:50, among other possibilities. In some embodiments, the first frequency is in a range from about 2 Hz to about 150 Hz and the second frequency is in a range from about 200 Hz to about 1000 Hz. In some embodiments, the first frequency is in a range from about 40 to about 90 Hz and the second frequency is in a range from about 200 Hz to about 1000 Hz. In some embodiments, in some embodiments, the first frequency is in a range from about 40 to about 60 Hz and the second frequency is in a range from about 400 Hz to about 1000 Hz. In some embodiments, the first frequency is in a range from about 40 to about 60 Hz and the second frequency is in a range from about 400 Hz to about 600 Hz.

In some embodiments, initiating delivery of the first electrical stimulation signal and the second electrical stimulation signal, ceasing delivery of the first electrical stimulation signal and the second electrical stimulation signal, altering a program parameter (e.g., duty cycle, pulse width, etc.) of the first electrical stimulation signal and/or the second electrical stimulation signal, or any combination thereof, can be based on accelerometer data (e.g., from an accelerometer in or coupled to the IPG), can be based on a time of day (e.g., whether it is nighttime or daytime for the patient), and/or can be based on an input from a patient (e.g., from a patient programmer) or clinician (e.g., from a clinician programmer), as is conventionally known. For example, postural changes or certain activities may affect where the lead contacts are stimulating. When a postural change, or a change in activity level is sensed (such as by use of an accelerometer positioned in the IPG), a parameter (e.g., duty cycle) can be altered (e.g., automatically by the IPG) to maintain desirable and effective stimulation. As a further example, the patient may wish to decrease stimulation strength while they sleep. The IPG system may be configured to adjust stimulation parameters at certain times of the day automatically and/or responsive to an input (e.g., a patient or clinician input). In some embodiments, the patient is able to adjust stimulation parameters with the patient programmer or remote control. For instance, the patient may be able to execute programs set by a physician.

In some embodiments, the pulses of the first stimulation can have a different (e.g., larger) area than an area of the pulses of the second stimulation. For instance, as illustrated in FIG. 5F, each of the respective pulses of the first stimulation can have an area that is larger than an area of the pulses of the second stimulation. In some examples, pulse shapes of the first electrical stimulation signal and the second electrical stimulation signal can be the same shape (e.g., biphasic square waves with active recovery). The pulses of the first electrical stimulation signal and the second electrical stimulation signal may be square waves, ramped square waves, triangular outputs, semi-sinusoidal, or sinusoidal outputs. In some embodiments, the pulse shape may feature of a shape, such as increasing or decreasing a slope. In some embodiments, the pulses for each of the waveforms can be biphasic pulses where each stimulation pulse is biphasic, meaning it comprises a first pulse phase followed essentially immediately thereafter by an opposite polarity pulse phase. Pulse shape can be modified, if non-square waves are available, and changes can be made to burst parameters, ramping up/down, or any other desired parameters of stimulation. Repeated stimulation, sensing and adjustments to various stimulation parameters may be made if necessary to target the desired treatment area and stimulation strength.

In some embodiments, arbitration (e.g., delay initiation or ceasing further delivery) of one or both of the first stimulation and the second stimulation can, optionally, be performed. For instance, one or both of the first stimulation and the second stimulation can be arbitrated (e.g., by three milliseconds, etc.) to ensure that a desired duty cycle (e.g., between “ON” and “OFF” time periods, as described herein) is maintained. For example, arbitration (3 ms of arbitration) ensures that area A or the ON time period 71 takes precedence so Area B or the OFF time period 73 gets pushed back if two pulses overlap or interleave.

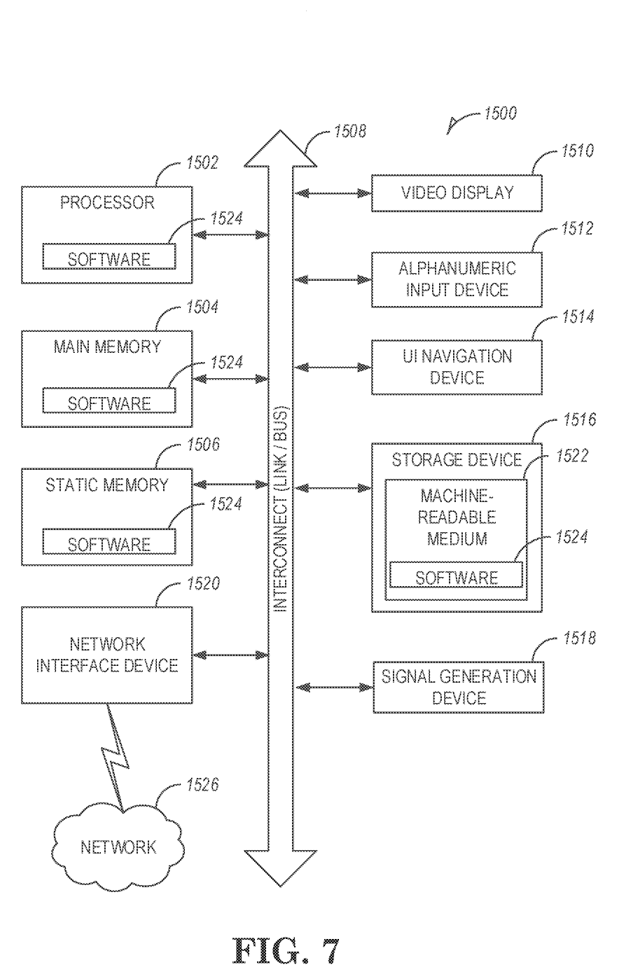

FIG. 7 is a block diagram illustrating a machine in the example form of a computer system 1500, within which a set or sequence of instructions may be executed to cause the machine to perform any one of the methodologies discussed herein pertaining to spinal pattern stimulation, according to an example embodiment. The machine or computer system 1500 may be used as a clinician programmer for programming an implantable pulse generator of FIG. 1B. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User’s Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1500 includes at least one processor 1502 (e.g., a central processing unit (CPU), a graphics processing unit (GPU) or both, processor cores, compute nodes, etc.), a main memory 1504 and a static memory 1506, which communicate with each other via a link 1508 (e.g., bus). The computer system 1500 may further include a video display unit 1510, an alphanumeric input device 1512 (e.g., a keyboard), and a user interface (UI) navigation device 1514 (e.g., a mouse). In one embodiment, the video display unit 1510, input device 1512 and UI navigation device 1514 are incorporated into a touch screen display. The computer system 1500 may additionally include a storage device 1516 (e.g., a drive unit), a signal generation device 1518 (e.g., a speaker), a network interface device 1520, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 7 (such as a GPU, video display unit, keyboard, etc.).

The storage device 1516 includes a machine-readable medium 1522 on which is stored one or more sets of data structures and instructions 1524 (e.g., software) embodying or utilized by any one or more of the methodologies or functions described herein. The instructions 1524 may also reside, completely or at least partially, within the main memory 1504, static memory 1506, and/or within the processor 1502 during execution thereof by the computer system 1500, with the main memory 1504, static memory 1506, and the processor 1502 also constituting machine-readable media.

While the machine-readable medium 1522 is illustrated in an example embodiment to be a single medium, the term “machine-readable medium” may include a single medium or multiple media (e.g., a centralized or distributed database, and/or associated caches and servers) that store the one or more instructions 1524. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1524 may further be transmitted or received over a communications network 1526 using a transmission medium via the network interface device 1520 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method for providing electrical stimulation to a patient’s spinal cord to treat at least inflammatory pain using an IPG system comprising one or more leads implanted in the patient’s spinal column, each lead comprising a plurality of electrodes, the method comprising:
delivering a first electrical stimulation signal at a first frequency between about 2 Hertz (Hz) to about 150 Hz to a patient's spinal cord; and
delivering a second electrical stimulation signal at a second frequency between 200 Hz to about 1000 Hz to the patient's spinal cord concurrently with the first electrical stimulation signal.

2. The method of claim 1, wherein both the first and second electrical stimulation signals are delivered at amplitudes that are less than about 80 percent of a paresthesia threshold.

3. The method of claim 1, wherein both the first and second electrical stimulation signals are delivered at amplitudes between about 20 to about 80 percent of a paresthesia threshold.

4. The method of claim 1, wherein both the first and second electrical stimulation signals are delivered at amplitudes between about 20 to about 40 percent of a paresthesia threshold.

5. The method of claim 1, wherein a ratio of the first frequency to the second frequency is in a range from about 1:10 to about 1:50.

6. The method of claim 5, wherein the first frequency is in a range from about 20 Hz to about 90 Hz.

7. The method of claim 5, wherein the second frequency is in a range from about 400 Hz to about 600 Hz.

8. The method of claim 1, further comprising cycling the concurrent delivery of the first and second electrical stimulation signals at a duty cycle that is less than about 0.8.

9. The method of claim 1, further comprising cycling the concurrent delivery of the first and second electrical stimulation signals at a duty cycle in the range from about 0.3 to about 0.7.

10. The method of claim 1, further comprising cycling the concurrent delivery of the first and second electrical stimulation signals at a duty cycle in the range from about 0.3.

11. The method of claim 1, wherein a pulse width of the first electrical stimulation signal is larger than a pulse width of the second electrical stimulation signal.

12. The method of claim 11, wherein:
the first electrical stimulation signal has a pulse width between about 80 to about 200 microseconds; and
the second electrical stimulation signal has a pulse width between about 20 to about 80 microseconds.

13. The method of claim 1, wherein an area in the spinal cord stimulated by the first and second signals overlap a dermatome associated with the inflammatory pain.

14. The method of claim 1, further comprising initiating delivery of the first electrical stimulation signal and the second electrical stimulation signal, ceasing delivery of the first electrical stimulation signal and the second electrical stimulation signal, altering a parameter of the first electrical stimulation signal and the second electrical stimulation signal, or any combination thereof, based on:
accelerometer data;
a time of day;
a patient or clinician input; or
any combination thereof.

15. The method of claim 1, further comprising delivering the first electrical stimulation signal and the second electrical stimulation signals as active recharge electrical stimulation signals, as square waves, or both.

16. A spinal cord stimulation system comprising:
a lead including a plurality of electrodes; and
an implantable pulse generator (IPG) configured to:
generate a first electrical stimulation signal at a first frequency between about 2 to about 150 Hz and a pulse width between about 80 to about 200 microseconds;
generate a second electrical stimulation signal at a second frequency between 400-1000 Hz and a pulse width between about 20 to about 80 microseconds;
deliver, via the plurality of electrodes, the first electrical stimulation signal and the second electrical stimulation signal concurrently to a patient's spinal cord at amplitudes between about 20 to about 80 percent of a paresthesia threshold of the patient to treat at least inflammatory pain of the patient; and
cycle the concurrent delivery according to a duty cycle in a range from about 0.2 to about 0.8.

17. The spinal cord stimulation system of claim 16, wherein the IPG is configured to
deliver, via at least four of the plurality of electrodes, the first electrical stimulation signal and the second electrical stimulation signal concurrently.

18. The spinal cord stimulation system of claim 17, wherein:
the at least four electrodes includes at least two cathodic electrodes and at least two anodic electrodes; and
the at least two cathodic electrodes are spaced at least one electrode away from the at least two anodic electrodes.

19. A non-transitory computer-readable medium having instructions stored thereon to cause circuitry in a computing device to:
generate a first electrical stimulation signal at a first frequency between about 2 to about 150 Hz and a pulse width between about 80 to about 200 microseconds;
generate a second electrical stimulation signal at a second frequency between 400-600 Hz and a pulse width between about 20 to about 80 microseconds;
deliver the first electrical stimulation signal and the second electrical stimulation signal concurrently to a spinal cord of a patient at amplitudes between about 20 to about 40 % of a paresthesia threshold of the patient to treat mixed pain of the patient, wherein a delivery location of the first electrical signal and a delivery location of the second electrical signal overlap and are configured to target the same dermatome associated with the mixed pain; and
cycle the concurrent delivery according to a duty cycle in a range from about 0.2 to about 0.8.

20. The non-transitory computer-readable medium of claim 19, wherein:
the first electrical stimulation signal and the second electrical stimulation signal are configured as a series of square wave pulses with active refresh; and
the first electrical stimulation signal and the second electrical stimulation signal are delivered for two hours or less per day.
